(19) United States
(12) Patent Application Publication
(10) Pub. No.: US 2014/0303605 A1
(43) Pub. Date: Oct. 9, 2014

(54) ACTIVE TREMOR CONTROL IN SURGICAL INSTRUMENTS RESPONSIVE TO A PARTICULAR USER

(71) Applicant: Elwha LLC, (US)

(72) Inventors: Edward S. Boyden, Chestnut Hill, MA (US); Gregory J. Della Rocca, Columbia, MO (US); Roderick A. Hyde, Redmond, WA (US); Robert Langer, Newton, MA (US); Eric C. Leuthardt, St. Louis, MO (US); Terence Myckatyn, St. Louis, MO (US); Parag Jitendra Parikh, St. Louis, MO (US); Dennis J. Rivet, Chesapeake, VA (US); Joshua S. Shimony, St. Louis, MO (US); Michael A. Smith, Phoenix, AZ (US); Clarence T. Tegreene, Mercer Island, WA (US)

(21) Appl. No.: 13/857,044

(22) Filed: Apr. 4, 2013

(51) Int. Cl. A61B 17/00

(52) U.S. Cl. CPC A61B 17/00; A61B 2017/00207
USPC 606/1

(57) ABSTRACT

Described embodiments include a method. The method includes initiating a hand tremor characterization mode of a handheld or hand operated surgical instrument having a working tip. The method includes detecting a hand tremble motion imparted by a user during the characterization mode. The method includes recognizing a pattern in the detected hand tremble motion and saving the recognized pattern in a computer storage media. The method includes initiating a hand tremor suppression mode of the surgical instrument. The method includes detecting a hand tremble motion in the surgical instrument imparted by a user during the suppression mode. The method includes retrieving the recognized pattern from the computer storage media. The method includes stabilizing the working tip by activating an actuator, the activation responsive to the recognized pattern and to the detected user-imparted hand tremble motion during the suppression mode.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)). In addition, the present application is related to the “Related Applications,” if any, listed below.

### PRIORITY APPLICATIONS

None.

### RELATED APPLICATIONS

U.S. patent application Ser. No. To Be Assigned, entitled ACTIVE TREMOR CONTROL IN SURGICAL INSTRUMENTS, naming Edward S. Boyden, Gregory J. Della Rocca, Roderick A. Hyde, Robert Langer, Eric C. Leuthardt, Terence Myckatyn, Parag Jitendra Parikh, Dennis J. Rivet, Joshua S. Shimony, Michael A. Smith, and Clarence T. Tegreene as inventors, filed 4, Apr. 2013 with attorney docket no. 0411-002-014-000000, is related to the present application.

If the listings of applications provided above are inconsistent with the listings provided via an ADS, it is the intent of the Applicant to claim priority to each application that appears in the Priority Applications section of the ADS and to each application that appears in the Priority Applications section of this application.

All subject matter of the Priority Applications and the Related Applications and of any and all parent, grandparent, great-grandparent, etc. applications of the Priority Applications and the Related Applications, including any priority claims, is incorporated herein by reference to the extent such subject matter is not inconsistent herewith.

### SUMMARY

For example, and without limitation, an embodiment of the subject matter includes a handheld or hand operated surgical instrument. The surgical instrument includes an elongated member having a longitudinal axis and a handle portion configured to be gripped or held by a user. The surgical instrument includes a working tip coupled to the elongated member. The surgical instrument includes a sensor configured to detect a hand tremble motion in the elongated member of the surgical instrument imparted by the user. The surgical instrument includes a mode controller configured to receive a selection of a hand tremor characterization mode of the surgical instrument or a hand tremor suppression mode of the surgical instrument. The surgical instrument includes a pattern recognition module configured to recognize a pattern in the detected hand tremble motion in the elongated member imparted by the user if the hand tremor characterization mode is selected. The surgical instrument includes a flexible beam element of the elongated member configured to reversibly bend, extend, or rotate with respect to the longitudinal axis. The surgical instrument includes an actuator physically coupled to the flexible beam element and configured to reversibly bend, extend, or rotate the flexible beam element with respect to the longitudinal axis. The surgical instrument includes a controller configured to stabilize the working tip by activating the actuator if the hand tremor suppression mode is selected. The activating is responsive to the recognized pattern and to the detected user-imparted hand tremble motion during the suppression mode. In an embodiment, the surgical instrument includes a computer storage media configured to save the recognized pattern in the user-imparted hand tremble motion.

For example, and without limitation, an embodiment of the subject matter described herein includes a method. The method includes initiating a hand tremor characterization mode of a handheld or hand operated surgical instrument having a working tip. The method includes detecting a hand tremble motion in the surgical instrument imparted by a user during the characterization mode. The method includes recognizing a pattern in the detected hand tremble motion. The method includes saving the recognized pattern in a computer storage media. The method includes initiating a hand tremor suppression mode of the surgical instrument. The method includes detecting a hand tremble motion in the surgical instrument imparted by a user during the suppression mode. The method includes retrieving the recognized pattern from the computer storage media. The method includes stabilizing the working tip by activating an actuator, the activation responsive to the recognized pattern and to the detected user-imparted hand tremble motion during the suppression mode.

For example, and without limitation, another embodiment of the subject matter described herein includes a method of characterizing a hand tremor motion created by a particular user in a handheld or hand operated surgical instrument. The method includes initiating a hand tremor characterization mode of a handheld or hand operated surgical instrument having a working tip. The method includes detecting a hand tremble motion in the surgical instrument imparted by a user during the characterization mode. The method includes recognizing a pattern in the detected user-imparted hand tremble motion. The method includes saving the recognized pattern in a computer storage media. In an embodiment, the method includes issuing a warning if the recognized pattern exceeds a threshold level of tremor severity. In an embodiment, the method includes inactivating an aspect of the surgical instrument if the recognized pattern exceeds a threshold level of tremor severity. In an embodiment, the method includes receiving an identifier of the particular user.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 illustrates an example environment 200 in which embodiments may be implemented;

FIG. 2 illustrates an example bending actuator of the surgical instrument of FIG. 1;

FIG. 3 illustrates an example pattern recognition module of the surgical instrument 205 of FIG. 1;

FIG. 4 illustrates an alternative embodiment of the handheld or hand operated surgical instrument 205 of FIG. 1;

FIG. 5 illustrates another alternative embodiment of the handheld or hand operated surgical instrument 205 of FIG. 1;

FIG. 6 illustrates an example operational flow stabilizing a working tip of surgical instrument;

FIG. 7 illustrates an example handheld or hand operated surgical instrument 505;

FIG. 8 illustrates an operational flow 600;

FIG. 9 illustrates an example operational flow 700 of characterizing a hand tremor motion created by a particular user in a handheld or hand operated surgical instrument; and

FIG. 10 illustrates an example operational flow 800 of stabilizing a working tip of a handheld or hand operated surgical instrument with respect to a hand tremor motion created by a particular user.

### DETAILED DESCRIPTION

In the following detailed description, reference is made to the accompanying drawings, which form a part hereof In the drawings, similar symbols typically identify similar components, unless context dictates otherwise. The illustrated embodiments described in the detailed description, drawings, and claims are not meant to be limiting. Other embodiments may be utilized, and other changes may be made, without departing from the spirit or scope of the subject matter presented here.

Those having skill in the art will recognize that the state of the art has progressed to the point where there is little distinction left between hardware, software, and/or firmware implementations of aspects of systems; the use of hardware, software, and/or firmware is generally (but not always, in that in certain contexts the choice between hardware and software can become significant) a design choice representing cost vs. efficiency tradeoffs. Those having skill in the art will appreciate that there are various implementations by which processes and/or systems and/or other technologies described herein can be effected (e.g., hardware, software, and/or firmware), and that the preferred implementation will vary with the context in which the processes and/or systems and/or other technologies are deployed. For example, if an implementer determines that speed and accuracy are paramount, the implementer may opt for a mainly hardware and/or firmware implementation; alternatively, if flexibility is paramount, the implementer may opt for a mainly software implementation; or, yet again alternatively, the implementer may opt for some combination of hardware, software, and/or firmware. Hence, there are several possible implementations by which the processes and/or devices and/or other technologies described herein may be effected, none of which is inherently superior to the other in that any implementation to be utilized is a choice dependent upon the context in which the implementation will be deployed and the specific concerns (e.g., speed, flexibility, or predictability) of the implementer, any of which may vary. Those skilled in the art will recognize that optical aspects of implementations will typically employ optically-oriented hardware, software, and or firmware.

In some implementations described herein, logic and similar implementations may include software or other control structures suitable to implement an operation. Electronic circuitry, for example, may manifest one or more paths of electrical current constructed and arranged to implement various logic functions as described herein. In some implementations, one or more media are configured to bear a device-detectable implementation if such media hold or transmit a special-purpose device instruction set operable to perform as described herein. In some variants, for example, this may manifest as an update or other modification of existing software or firmware, or of gate arrays or other programmable hardware, such as by performing a reception of or a transmission of one or more instructions in relation to one or more operations described herein. Alternatively or additionally, in some variants, an implementation may include special-purpose hardware, software, firmware components, and/or general-purpose components executing or otherwise invoking special-purpose components. Specifications or other implementations may be transmitted by one or more instances of tangible transmission media as described herein, optionally by packet transmission or otherwise by passing through distributed media at various times.

Alternatively or additionally, implementations may include executing a special-purpose instruction sequence or otherwise invoking circuitry for enabling, triggering, coordinating, requesting, or otherwise causing one or more occurrences of any functional operations described below. In some variants, operational or other logical descriptions herein may be expressed directly as source code and compiled or otherwise invoked as an executable instruction sequence. In some contexts, for example, C++ or other code sequences can be compiled directly or otherwise implemented in high-level descriptor languages (e.g., a logic-synthesizable language, a hardware description language, a hardware design simulation, and/or other such similar mode(s) of expression). Alternatively or additionally, some or all of the logical expression may be manifested as a Verilog-type hardware description or other circuitry model before physical implementation in hardware, especially for basic operations or timing-critical applications. Those skilled in the art will recognize how to obtain, configure, and optimize suitable transmission or computational elements, material supplies, actuators, or other common structures in light of these teachings.

In a general sense, those skilled in the art will recognize that the various embodiments described herein can be implemented, individually and/or collectively, by various types of electro-mechanical systems having a wide range of electrical components such as hardware, software, firmware, and/or virtually any combination thereof; and a wide range of components that may impart mechanical force or motion such as rigid bodies, spring or torsional bodies, hydraulics, electro-magnetically actuated devices, and/or virtually any combination thereof. Consequently, as used herein “electro-mechanical system” includes, but is not limited to, electrical circuitry operably coupled with a transducer (e.g., an actuator, a motor, a piezoelectric crystal, a Micro Electro Mechanical System (MEMS), etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, module, communications switch, optical-electrical equipment, etc.), and/or any non-electrical analog thereto, such as optical or other analogs. Those skilled in the art will also appreciate that examples of electro-mechanical systems include but are not limited to a variety of consumer electronics systems, medical devices, as well as other systems such as motorized transport systems, factory automation systems, security systems, and/or communication/computing systems. Those skilled in the art will recognize that electro-mechanical as used herein is not necessarily limited to a system that has both electrical and mechanical actuation except as context may dictate otherwise.

In a general sense, those skilled in the art will also recognize that the various aspects described herein which can be implemented, individually and/or collectively, by a wide range of hardware, software, firmware, and/or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), and/or electrical circuitry forming a communications device (e.g., a modem, communications switch, optical-electrical equipment, etc.). Those having skill in the art will recognize that the subject matter described herein may be implemented in an analog or digital fashion or some combination thereof.

Computing system environments typically includes a variety of computer-readable media products. Computer-readable media may include any media that can be accessed by a computing device and include both volatile and nonvolatile media, removable and non-removable media. By way of example, and not of limitation, computer-readable media may include computer storage media. By way of further example, and not of limitation, computer-readable media may include a communication media.

Computer storage media includes volatile and nonvolatile, removable and non-removable media implemented in any method or technology for storage of information such as computer-readable instructions, data structures, program modules, or other data. Computer storage media includes, but is not limited to, random-access memory (RAM), read-only memory (ROM), electrically erasable programmable read-only memory (EEPROM), flash memory, or other memory technology, CD-ROM, digital versatile disks (DVD), or other optical disk storage, magnetic cassettes, magnetic tape, magnetic disk storage, or other magnetic storage devices, or any other medium which can be used to store the desired information and which can be accessed by a computing device. In a further embodiment, a computer storage media may include a group of computer storage media devices. In another embodiment, a computer storage media may include an information store. In another embodiment, an information store may include a quantum memory, a photonic quantum memory, or atomic quantum memory. Combinations of any of the above may also be included within the scope of computer-readable media.

Communication media may typically embody computer-readable instructions, data structures, program modules, or other data in a modulated data signal such as a carrier wave or other transport mechanism and include any information delivery media. The term “modulated data signal” means a signal that has one or more of its characteristics set or changed in such a manner as to encode information in the signal. By way of example, and not limitation, communications media may include wired media, such as a wired network and a direct-wired connection, and wireless media such as acoustic, RF, optical, and infrared media.

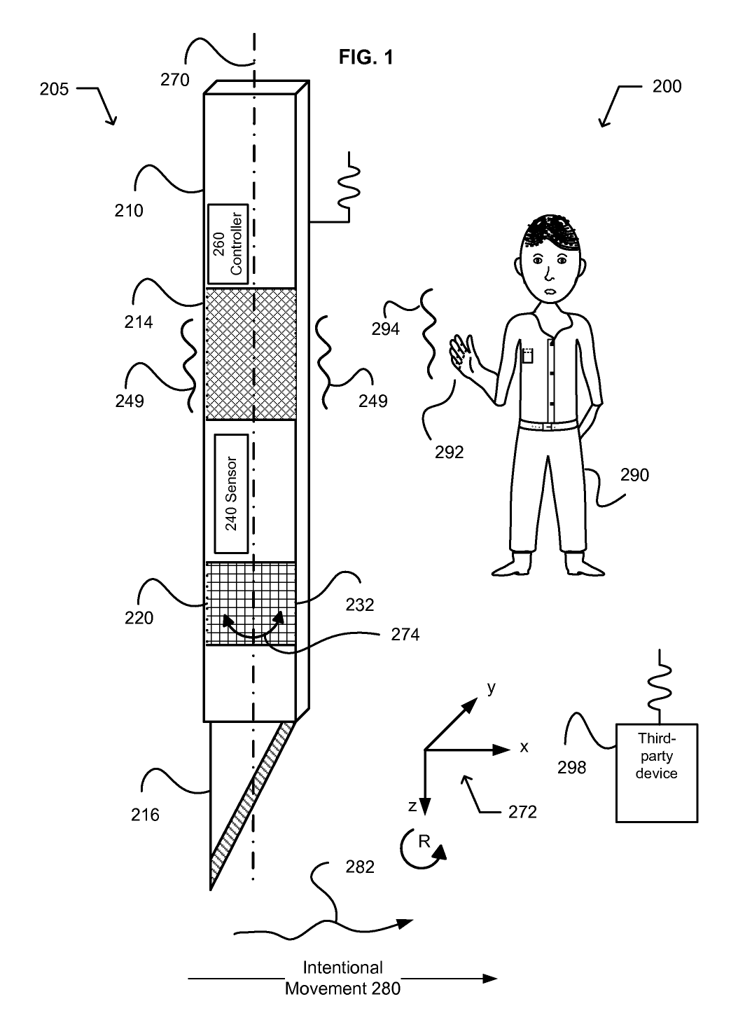

FIG. 1 illustrates an example environment 200 in which embodiments may be implemented. The environment includes a handheld or hand operated surgical instrument 205, and a user 290 of the surgical instrument. The user includes a hand 292 or other extremity suitable for gripping or holding the surgical instrument. The environment also includes a third-party device 298 configured to communicate with the surgical instrument. For example, the third-party device and the surgical instrument may communicate wirelessly, such as by Bluetooth or other wireless protocol. In an embodiment, the handheld or hand operated surgical instrument may include a manual surgical instrument for general use, such as a non-powered, hand-held, or hand-manipulated device, either reusable or disposable, intended to be used in various general surgical procedures as described in 21 C.F.R. 878.4800. In an embodiment, the handheld or hand operated surgical instrument may include a powered hand-held or hand-manipulated device.

The handheld or hand operated surgical instrument 205 includes an elongated member 210 having a longitudinal axis 270 and a handle portion 214 configured to be gripped or held by the extremity 292 of the user 290. The surgical instrument includes a working tip 216 coupled to the elongated member. The surgical instrument includes a flexible beam element 220 of the elongated member located in-between the handle portion and the working tip 213. The flexible beam element is configured to reversibly bend 274 with respect to the longitudinal axis. For example, FIG. 1 illustrates the reversible bend 274 being in the x-z plane or about the y axis of 272. In an embodiment, the flexible beam element is located in-between the handle portion and the working tip. The surgical instrument includes a bending actuator 232 physically coupled to the flexible beam element and configured to reversibly bend the flexible beam element. In an embodiment, the flexible beam element includes a flexible beam system.

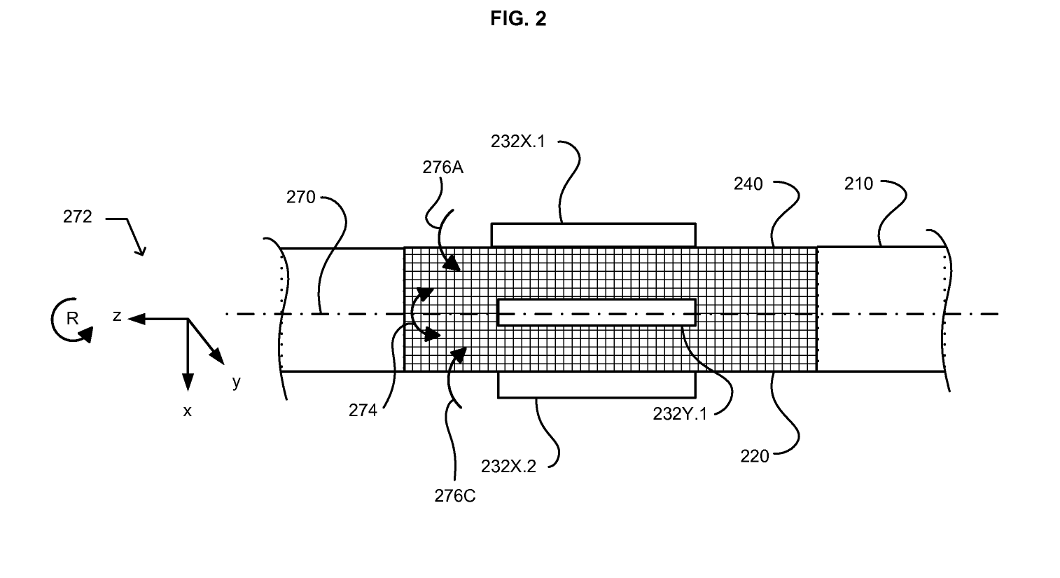

An embodiment of the bending actuator 232 is illustrated in FIG. 2 by a bending actuator 232X.1 configured to bend in the x-z plane as illustrated by a bending motion 276A. One or more additional bending actuators may be included in the flexible beam element 220. For example, a bending actuator 232X.2 may be configured to bend in the x-z plane as illustrated by a bending motion 276C and opposite to bending motion 276A. Alternatively, the bending actuators 232X.1 and 232X.2 may be configured to cooperate in creating a bending motion in the x-z plane, such as cooperatively creating the bending motion 274. In an embodiment, the bending actuator 232X.1 and two additional bending actuators may be spaced 120° apart around the flexible beam element to provide a range of movements in both x-z and y-z planes. In an embodiment of the bending actuator 232, a bending actuator 232Y.1 is configured to bend the flexible beam element in the y-z plane. An example bending actuator may include a Piezo Bender Actuator manufactured by PI (Physik Instrumente) of Karlsruhe, Germany. In an embodiment, one or more of the bending actuators may be wholly within the flexible beam element. In an embodiment, a bending actuator may be attached at one end to a non-bending portion of the elongated member and to the flexible beam element at another end. In an embodiment, a bending actuator may span the flexible beam element and each end attached to non-bending portion of the elongated member.

Returning to FIG. 1, the handheld or hand operated surgical instrument 205 includes a sensor 240 configured to detect a user-imparted hand tremble motion 249 of the elongated member 210. For example, the user-imparted hand tremble motion may be created by a tremor-shaking movement 294 by the appendage 292 of the user 290 occurring during a purposeful movement, at rest, or holding a position against gravity. For example, the user-imparted hand tremble motion may be created by fatigue, caffeine or stimulants, lack of practice, or age. For example, the user-imparted hand tremble motion may include a physiological tremor. For example, the user-imparted hand tremble motion may have a frequency between 1 or 2 to about 15 Hz. A consequence of the user-imparted hand tremble motion may be to impart the tremble motion into the working tip 216 and change a user intentional movement 280 into a trembling movement 282. In an embodiment, the sensor is configured to detect a user-imparted hand tremble motion or non-tremulous error. The surgical instrument includes a controller 250 configured to stabilize the working tip 216 by activating the bending actuator 232 in response to the detected user-imparted hand tremble motion 249.

In an embodiment, the working tip 216 includes a tissue cutting device. For example a tissue cutting device may include a surgical blade, a saw, or a drill. In an embodiment, the working tip includes an electro-cautery device. In an embodiment, the working tip includes a tissue fixation device. In an embodiment, the working tip includes an effector. In an embodiment, the flexible beam element 220 of the elongated member 210 is configured to reversibly bend with respect to the longitudinal axis with one degree of freedom. For example, one degree of freedom may include reversibly bending with respect to the x-z plane of the longitudinal axis.

In an embodiment, the flexible beam element 220 of the elongated member 210 is configured to reversibly bend 274 with respect to the longitudinal axis 270 with two degrees of freedom. For example, two degrees of freedom may include reversibly bending with respect to the x-z plane and the y-z plane of the longitudinal axis.

In an embodiment, the bending actuator 232 includes two bending actuators orthogonally orientated to each other. FIG. 2 illustrates an embodiment where the bending actuator 232X.1 and the bending actuator 232Y.1 are orthogonally orientated to each other. In an embodiment, the flexible beam element 220 and the bending actuator include a piezoelectric cantilever structure. In an embodiment, the bending actuator includes a piezoelectric bending actuator. For example, the bending actuator may include a micro piezoelectric bending actuator. In an embodiment, the bending actuator includes a piezoelectric strip actuator. In an embodiment, the bending actuator includes a piezoelectric bimorph actuator. In an embodiment, the bending actuator includes a piezoelectric multimorph actuator. In an embodiment, the bending actuator includes a piezoelectric patch actuator. In an embodiment, the bending actuator includes a magnetostricitive actuator. In an embodiment, the bending actuator includes a shape memory actuator. In an embodiment, the bending actuator is disposed on or bonded to the flexible beam element. In an embodiment, the bending actuator is configured to reversibly deform the flexible beam element.

In an embodiment, the sensor 240 is configured to measure a stress or strain at one or more longitudinal positions or locations along the elongated member 210. In an embodiment, the sensor is configured to measure a stress or strain at a pair of sensors located at opposing lateral positions on a plane orthogonal to the longitudinal axis 270 of the elongated member. Such a pair of measurements can provide data both on bending about the longitudinal axis and on extension/contraction along the longitudinal axis. In an embodiment, the sensor is configured to measure a stress or strain at two or more lateral positions at a plane orthogonal to the longitudinal axis of the elongated member. In an embodiment, the sensor is configured to measure a differential stress or strain at two or more lateral positions at a plane orthogonal to the longitudinal axis of the elongated member, thereby providing data on bending about the longitudinal axis. In an embodiment, the sensor is configured to measure a bending moment at one or more longitudinal positions along the elongated member.

In an embodiment, the sensor 240 includes a piezoelectric sensor. In an embodiment, a piezoelectric bending actuator 232 includes the piezoelectric sensor. For example, the sensor may be integrated into an actuator, as in a piezo patch. In an embodiment, the sensor includes a sensor on-board the surgical instrument. In an embodiment, the sensor includes a sensor internally referenced to the surgical instrument 205. In an embodiment, the sensor includes a MEMS sensor. In an embodiment, the sensor includes an interferometric sensor, which may incorporate an optical fiber. In an embodiment, the sensor includes an optical fiber strain sensor. For example, optical fiber strain sensors are described in Sylvie Delepine-Lesoille, et al., Optical fiber strain sensors for use in civil engineering, 272 BLPC 123 (October/November 2008). In an embodiment, the sensor includes an accelerometer. For example, the accelerometer may include a one to a four axis accelerometer. In an embodiment, the sensor includes a gyroscope, such as a MEMS gyroscope, a ring laser gyroscope, or an optical fiber gyroscope. For example, a MEMS gyroscope is marketed by Silicon Sensing System Japan as MEMS silicon ring gyro CRS03. (http://www.sssj.co.jp/en/products/gyro/crs03.html, accessed Apr. 2, 2013). For example, ring laser and fiber optic gyroscope are described by Jeng-Nan Juang and R. Radharamanan, Evaluation of ring laser and fiber optic gyroscope technology, (https://docs.google.com/viewer?a=v&q=cache:PNuEILz6u-0J:www.asee.org/documents/sections/middle-atlantic/fall-2009/01-Evaluation-Of-Ring-Laser-And-Fiber-Optic-Gyroscope-Technology.pdf+&hl=en&gl=us&pid=bl&srcid=ADGEESjynJzDJ74kNLuHIHJmNU5k27p2u2 Va0N15a_ug0SRkTlwEQC-zxAB_{—}6hA2HES7ZmgF4VR0EI-U28W4g0NNdaLO6dwGJZiCmAoAHI4-1f03UCBqw0siuSyzMyGLASeqQ4sl0cNk&sig=AHIEtbRenMriWLR3vfnmXPnpmubjXvL6Sw (accessed Apr. 2, 2013)). For example, an accelerometer or gyroscope may be located within the handle portion 214 to detect the tremor directly, or within the elongated member 210 or at the working tip 216 to measure the net motion: tremble and actuator imparted.

In an embodiment, the controller 260 includes a closed loop controller. In an embodiment, the closed loop controller includes a recursive filter. For example, a recursive filter may include a Kalman filter. For example, the recursive filter may be used in developing a stabilization response. In an embodiment, the stabilization of the working tip 216 includes changing a tremble mode of the elongated member 210 and working tip system. In an embodiment, the stabilization of the working tip includes changing a modal frequency of the longitudinal member. For example, a modal frequency may be changed by changing a stiffness of the flexible beam element 220. In an embodiment, the controller includes a controller configured to stabilize the working tip 216 by suppressing a selected frequency component of the detected user-imparted hand tremble motion 249. In an embodiment, the controller includes a controller configured to stabilize the working tip by suppressing a selected magnitude of a motion of the detected user-imparted hand tremble motion. In an embodiment, the controller includes a controller configured to stabilize the working tip by suppressing a user-selected magnitude of a tremble motion component of the detected user-imparted hand tremble motion. In an embodiment, the controller is user-activatable or user-deactivatable. For example, the controller is operable only when actually working on patient, not before or after. In an embodiment, the controller is configured to stabilize the working tip with respect to the detected user-imparted hand tremble motion by activating the bending actuator 232 in response to the detected user-imparted hand tremble motion. In an embodiment, the controller is configured to stabilize the working tip with respect to the detected user-imparted hand tremble motion by activating the bending actuator 232 in response to the detected user-imparted hand tremble motion and counteract the detected user-imparted hand tremble motion.

In an embodiment, the controller 260 includes a controller configured to stabilize the working tip 216 by activating the bending actuator 232 in a manner suppressing a particular frequency component of the tremor in the detected user-imparted hand tremble motion 249. For example, techniques for active control of vibration in a flexible beam are described in Mohd S. Saad, et al., Active vibration control of flexible beam systems using proportional control scheme in finite difference simulation platform, 4th International Conference on Modeling, Simulation and Applied Optimization (ICMSAO) (2011). For example, techniques for active control of vibration are described in Cheol Song, et al., Active tremor cancellation by a “Smart” handheld viteoretinal microsurgical tool using swept source optical coherence tomography, 20 Optics Express 23414 (October 2012). For example, techniques for active control of vibration are described in Robert H. Cannon, Jr. and Eric Schmitz, Initial experiments on the end-point control of a flexible one-link robot, 3 The International Journal of Robotics Research 62 (1984). For example, techniques for active control of vibration are described in Mohd S. Saad, et al., Active vibration control of flexible beam using differential evolution optimization, 62 World Academy of Science, Engineering and Technology 419 (2012).

For example, the controller 260 may select a certain frequency range or tremor component of the detected user-imparted hand tremble motion 249 for suppression based upon a criteria or other standard. For example, the controller selects a certain frequency range or tremor component to suppress. For example, the controller selects a significant frequency range or tremor component to suppress. In an embodiment, the controller includes a controller configured to stabilize the working tip 216 by activating the bending actuator 232 in a manner suppressing a dynamically selected frequency component of the tremor in the detected user-imparted hand tremble motion. For example, a user's tremor frequency range may shift or broaden after they have been working awhile. The controller is configured to change its response accordingly. For example, a surgeon may start a procedure in good shape, but tire over time, and begin shaking after 20 minutes. For example, a magnitude of tremor may increase or a frequency component may shift over time after the user 290 has been working for 15 minutes. In an embodiment, the controller is further configured to increase control or increase suppression accordingly.

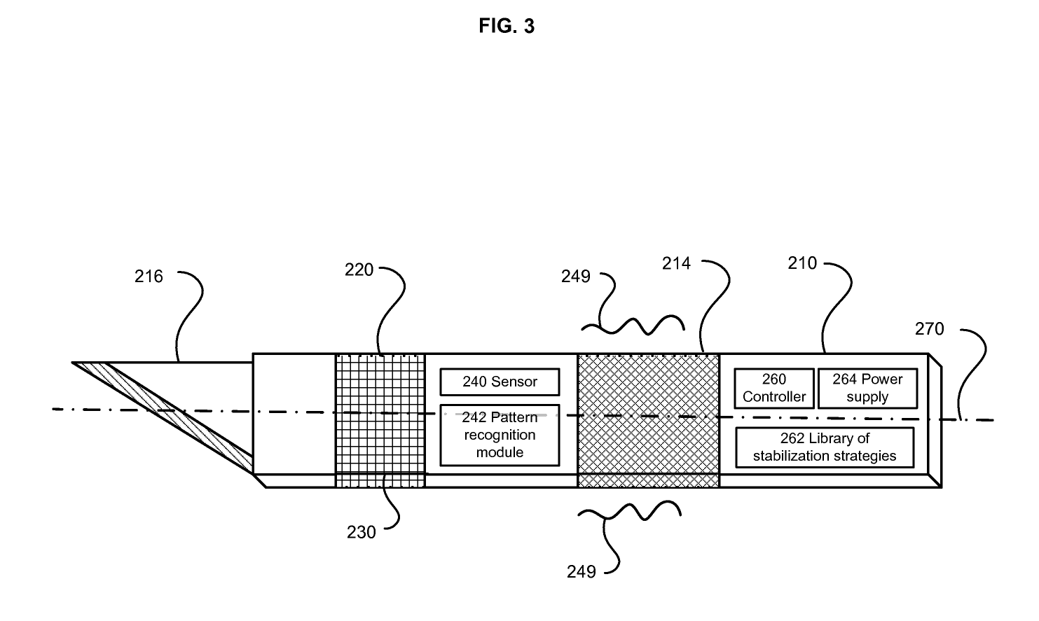

FIG. 3 illustrates an embodiment of the surgical instrument 205 of FIG. 1 that includes a pattern recognition module 242 configured to recognize a pattern in the detected user-imparted hand tremble motion 249 of the elongated member 210. For example, a recognized pattern may include a recognized pattern in the detected user-imparted hand tremble motion of the elongated member occurring over a period of time. For example, the recognized pattern may occur over a time period of one second, five second, ten seconds, 30 seconds, or a minute. For example, the recognized pattern may include a particular feature of the detected user-imparted hand tremble motion, such as a drift in the x plane or a persistent frequency, or such as a clockwise oscillation. In an embodiment, the controller 260 is configured to stabilize the working tip 216 by activating the bending actuator 232 in a manner responsive to the detected user-imparted hand tremble motion and to the recognized pattern in the detected user-imparted hand tremble motion 249. In an embodiment, the surgical instrument includes a power supply 264 suitable for powering the surgical instrument.

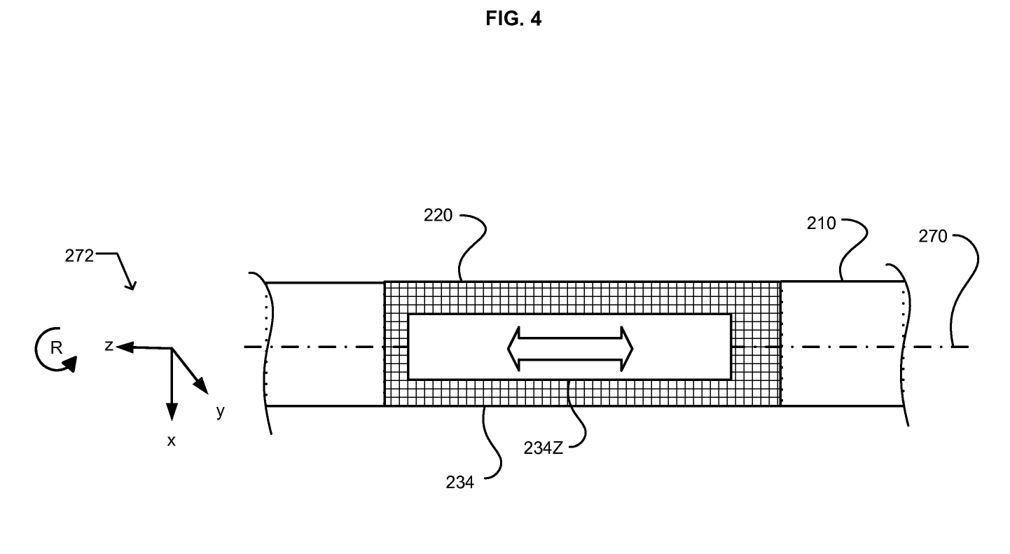

FIGS. 1 and 4 illustrate an alternative embodiment of the handheld or hand operated surgical instrument 205. In the alternative embodiment, the surgical instrument includes the elongated member 210 having the longitudinal axis 270, and having the handle portion 214 configured to be gripped or held by the user 290. The surgical instrument includes the working tip 216 coupled to the elongated member. The surgical instrument includes a flexible beam element 220 of the elongated member located in-between the handle portion and the working tip, and which is configured to reversibly lengthen or shorten along the longitudinal axis. The reversibly lengthen or shorten is illustrated in FIG. 4 as reversibly lengthen or shorten along the z axis of the four axis 272. A linear actuator 234Z embodiment of the linear actuator 234 is configured to reversibly lengthen or shorten the flexible beam element along the longitudinal axis. For example, the linear actuator may include a linear motor, linear piezomotor, extension motor, or multi-layer extension motor (stack actuator). The surgical instrument includes a sensor 240 configured to detect user-imparted hand tremble motion 249 of the elongated member. The surgical instrument includes a controller 260 configured to stabilize the working tip by activating the linear actuator in a manner responsive to the detected user-imparted hand tremble motion. In an embodiment, the linear actuator is physically coupled to at least a portion of the flexible beam element.

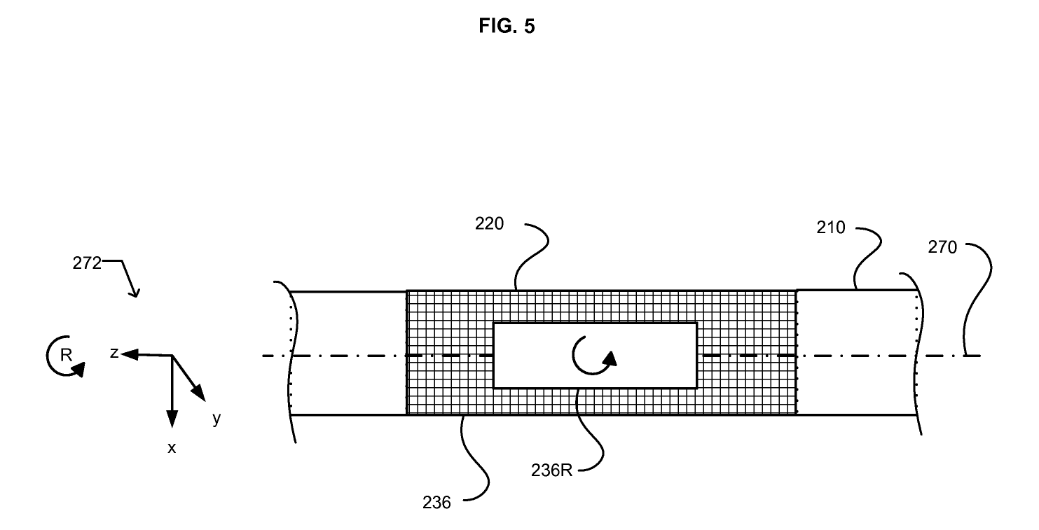

FIGS. 1 and 5 illustrate another alternative embodiment of the handheld or hand operated surgical instrument 205. In the alternative embodiment, the surgical instrument includes the elongated member 210 having the longitudinal axis 270, and having the handle portion 214 configured to be gripped or held by the user 290. The surgical instrument includes the working tip 216 coupled to the elongated member. The surgical instrument includes a flexible beam element 220 configured to reversibly rotate about the longitudinal axis. The surgical instrument includes a rotational actuator 236 physically coupled to the flexible beam element and configured to reversibly rotate a portion of the flexible beam element about the longitudinal axis. For example, the rotational actuator 236 is illustrated as a rotational actuator 236R. The surgical instrument includes a sensor 240 configured to detect user-imparted hand tremble motion 249 of the elongated member. The surgical instrument includes a controller 260 configured to stabilize the working tip by activating the rotational actuator in a manner reversibly rotating a portion of the flexible beam element about the longitudinal axis.

FIGS. 1-5 illustrate a further alternative embodiment of the handheld or hand operated surgical instrument 205. In the alternative embodiment, the surgical instrument 205 includes the elongated member 210 having the longitudinal axis 270 and a handle portion 214 configured to be gripped or held by the user 290. The surgical instrument includes the working tip 216 coupled to the elongated member. The surgical instrument includes a flexible beam element 220 of the elongated member located in-between the handle portion 214 and the working tip. The flexible beam element is configured to (i) reversibly bend with respect to the longitudinal axis, (ii) reversibly lengthen and shorten along the longitudinal axis, and (iii) reversibly rotate about the longitudinal axis. The surgical instrument includes the linear actuator 232 physically coupled to the flexible beam element and configured to reversibly bend the flexible beam element. The surgical instrument includes linear actuator 234Z physically coupled to the flexible beam element and configured to reversibly lengthen or shorten the flexible beam element. The surgical instrument includes the rotational actuator 236 physically coupled to the flexible beam element and configured to reversibly rotate a portion of the flexible beam element about the longitudinal axis. The surgical instrument includes the sensor 240 configured to detect user-imparted hand tremble motion 249 of the elongated member. The surgical instrument includes a controller configured to stabilize the working tip by (i) activating the bending actuator 232 in a manner reversibly bending the flexible beam element with respect to the longitudinal axis, (ii) activating the linear actuator in a manner reversibly lengthening and shortening the flexible beam element along the longitudinal axis, or (iii) activating the rotational actuator in a manner reversibly rotating a portion of the flexible beam element about the longitudinal axis.

FIGS. 1-5 illustrate aspects of a further alternative embodiment of the handheld or hand operated surgical instrument 205 for stabilizing the working tip 216 by suppressing a hand tremor motion imparted by the user 290. In the further alternative embodiment, the surgical instrument includes the elongated member 210 having the longitudinal axis 270 and a handle portion 214 configured to be gripped or held by the user 290. The surgical instrument includes the working tip 216 coupled to the elongated member. The surgical instrument includes the sensor 240 configured to detect a user-imparted hand tremble motion 249 of the elongated member. The surgical instrument includes a flexible beam element 220 of the elongated member located in-between the handle portion and the working tip. The flexible beam element is configured to reversibly bend, extend, or rotate with respect to the longitudinal axis. The surgical instrument includes an actuator physically coupled to the flexible beam element and configured to reversibly bend, extend, or rotate the flexible beam element with respect to the longitudinal axis. In an embodiment, the actuator may include the bending actuator 232, the linear actuator 234, or the rotational actuator 236. The surgical instrument includes the controller 260 configured to stabilize the working tip 216 by activating the actuator in a manner responsive to the detected user-imparted hand tremble motion 249 in at least one degree of freedom.

In an embodiment, the controller 260 includes a library 262 of at least two stabilization strategies. Each strategy of the at least two stabilization strategies is configured to stabilize the working tip 216 by suppressing a respective detected user-imparted hand tremble motion 249. In this embodiment, the controller 260 is configured to activate the actuator in accordance with a stabilization strategy responsive to the detected user-imparted hand tremble motion and selected from the at least two stabilization strategies. In an embodiment, the controller includes an algorithm specifying a manner of activating the activator to stabilize the working tip.

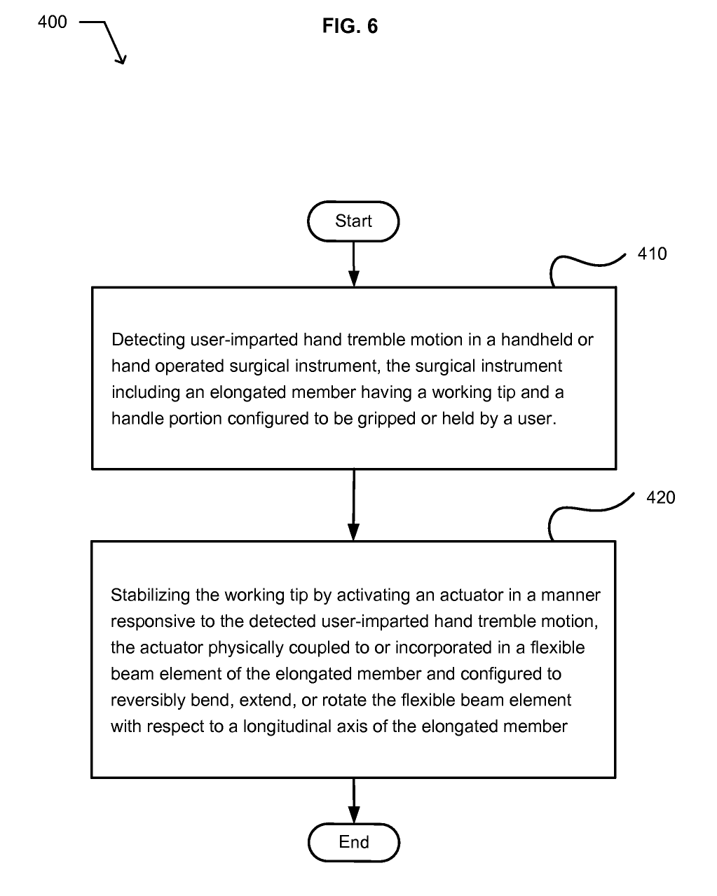

FIG. 6 illustrates an example operational flow 400 of stabilizing a working tip of a surgical instrument. After a start operation, the method includes a sensing operation 410. The sensing operation includes detecting user-imparted hand tremble motion in a handheld or hand operated surgical instrument. The surgical instrument includes an elongated member having a working tip and a handle portion configured to be gripped or held by a user. In an embodiment, the sensing operation may be implemented using the sensor 240 described in conjunction with FIGS. 1-5. A steadying operation 420 includes stabilizing the working tip by activating an actuator in a manner responsive to the detected user-imparted hand tremble motion. The actuator is physically coupled to or incorporated in a flexible beam element of the elongated member and is configured to reversibly bend, extend, or rotate the flexible beam element with respect to a longitudinal axis of the elongated member. In an embodiment, the steadying operation may be implemented using the controller 260 and at least one of the actuators 232, 234, or 236 described in conjunction with FIGS. 1-5. The operational flow includes an end operation.

In an embodiment of the steadying operation 420, the stabilizing of the working tip includes selecting a stabilization strategy responsive to the detected user-imparted hand tremble motion from a library of at least two stabilization strategies. Each strategy of the at least two stabilization strategies is configured to stabilize the working tip with respect to a respective detected user-imparted hand tremble motion. The steadying operation includes activating the actuator in compliance with the selected stabilization strategy. In an embodiment of the steadying operation, the stabilizing the working tip includes stabilizing the working tip by activating an actuator in accordance with an algorithm specifying the manner of activating the activator to stabilizing the working tip with respect to the detected user-imparted hand tremble motion. In an embodiment of the steadying operation, the stabilizing the working tip includes stabilizing the working tip with respect to the detected user-imparted hand tremble motion by activating an actuator in one degree of freedom and in a manner responsive to the detected user-imparted hand tremble motion. For example, one degree of freedom may include one of bending in an x-z plane with respect to the longitudinal axis, of bending in a y-z plane with respect to the longitudinal axis, extending along a z plane with respect to the longitudinal axis, or a rotation with respect to the longitudinal axis. In an embodiment of the steadying operation, the stabilizing the working tip includes stabilizing the working tip with respect to the detected user-imparted hand tremble motion by activating an actuator in two degrees of freedom and in a manner responsive to the detected user-imparted hand tremble motion. For example, two degrees of freedom may include any two of bending in an x-z plane with respect to the longitudinal axis, of bending in a y-z plane with respect to the longitudinal axis, extending along a z plane with respect to the longitudinal axis, or a rotation about the longitudinal axis. In an embodiment of the steadying operation, the stabilizing the working tip includes stabilizing the working tip with respect to the detected user-imparted hand tremble motion by activating an actuator in three degrees of freedom and in a manner responsive to the detected user-imparted hand tremble motion. For example, three degrees of freedom may include any three of bending in an x-z plane with respect to the longitudinal axis, of bending in a y-z plane with respect to the longitudinal axis, extending along a z plane with respect to the longitudinal axis, or a rotation with respect to the longitudinal axis. In an embodiment of the steadying operation, the stabilizing the working tip includes stabilizing the working tip with respect to the detected user-imparted hand tremble motion by activating an actuator in four degrees of freedom and in a manner responsive to the detected user-imparted hand tremble motion. In an embodiment of the steadying operation, the stabilizing the working tip may stabilize the working tip with respect to the detected user-imparted hand tremble motion by activating an actuator in one degree of freedom and in a manner responsive to an aspect of the detected user-imparted hand tremble motion.

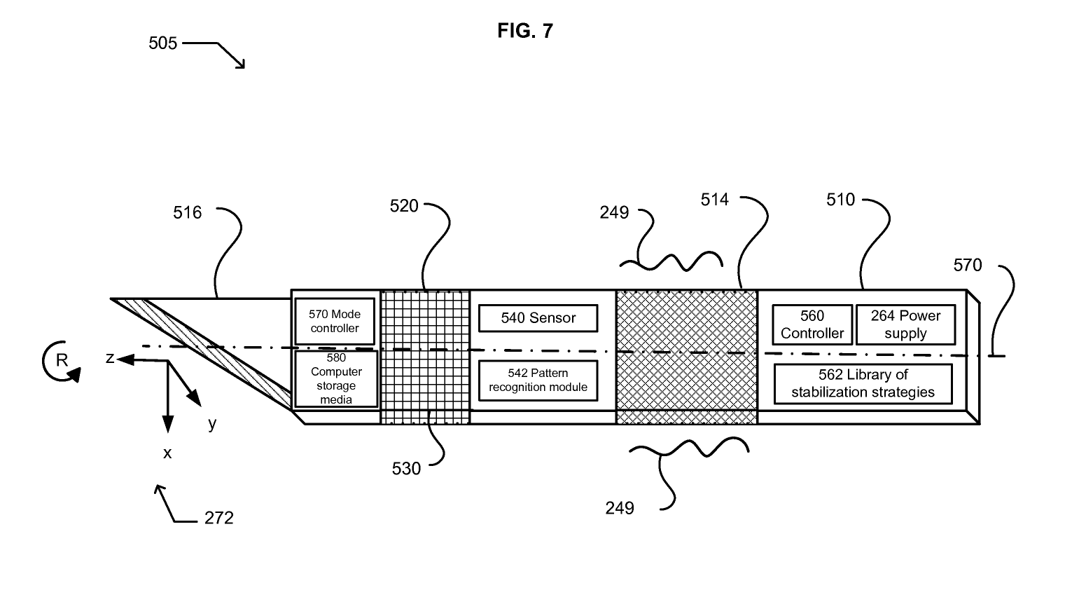

FIG. 7 illustrates a handheld or hand operated surgical instrument 505. The surgical instrument includes an elongated member 510 having a longitudinal axis 570 and a handle portion 514 configured to be gripped or held by a user, such as the user 290 described in conjunction with FIG. 1. The surgical instrument includes a working tip 516 coupled to the elongated member. The surgical instrument includes a sensor 540 configured to detect a hand tremble motion 249 in the elongated member of the surgical instrument imparted by the user 290. The surgical instrument includes a mode controller 570 configured to receive a selection of a hand tremor characterization mode of the surgical instrument or a hand tremor suppression mode of the surgical instrument. The surgical instrument includes a pattern recognition module 542 configured to recognize a pattern in the detected hand tremble motion 249 in the elongated member imparted by the user if the hand tremor characterization mode is selected. The surgical instrument includes a flexible beam element 520 of the elongated member. In an embodiment, the flexible beam element is located in-between the handle portion and the working tip. The flexible beam element and configured to reversibly bend, extend, or rotate with respect to the longitudinal axis. The surgical instrument includes an actuator 530 physically coupled to the flexible beam element. The actuator is configured to reversibly bend, extend, or rotate the flexible beam element with respect to the longitudinal axis. For example, in an embodiment, the actuator includes a bending actuator 232, such as described in conjunction with FIG. 2. For example, in an embodiment, the actuator includes a linear actuator 234, such as described in conjunction with FIG. 4. For example, in an embodiment, the actuator includes a rotational actuator 236, such as described in conjunction with FIG. 5. The surgical instrument includes a controller 560 configured to stabilize the working tip by activating the actuator if the hand tremor suppression mode is selected. The activating is responsive to the recognized pattern and to the detected user-imparted hand tremble motion during the suppression mode.

In an embodiment, the mode controller 570 is configured to receive a user selection of a hand tremor characterization mode of the surgical instrument 505 and an identifier of the user. In an embodiment, the pattern recognition module 542 is configured to associate the identifier of the user with the recognized pattern in the user-imparted hand tremble motion 249. In an embodiment, the pattern recognition module is configured to recognize a pattern in the detected hand tremble motion in the elongated member 510 imparted by the user occurring over a period of time. For example, the period of time may include one second, five seconds, 10 seconds, or 30 seconds. For example, the pattern may primarily include tremors in a distinctive frequency range, such as 5-8.5 Hz; this range can be used by the controller 560 to select the frequency response of the motion stabilization. In an embodiment, the controller 560 is further configured to control the activation of the actuator 530 in response to a combination of the recognized pattern in the user-imparted hand tremble motion and the detected user-imparted hand tremble motion. In an embodiment, the controller is further configured to control the activation of the actuator in response to a weighted combination of the recognized pattern in the user-imparted hand tremble motion and the detected user-imparted hand tremble motion.

In an embodiment, the surgical instrument 505 includes a computer storage media 580 configured to save the recognized pattern in the user-imparted hand tremble motion. In an embodiment, the computer storage media is configured to save the recognized pattern in the user-imparted hand tremble motion in an association with an identifier of the user. In an embodiment, the stabilization controller is further configured to retrieve from a computer storage media the recognized pattern in the user-imparted hand tremble motion saved in an association with an identifier of the user.

In an embodiment, the surgical instrument 505 is sterilized. In an embodiment, the surgical instrument is configured to be usable after sterilization. In an embodiment, the surgical instrument is configured to be usable after an exposure to one sterilization for a surgical use. In an embodiment, the surgical instrument is configured to be usable after an exposure to a surgical sterilization condition. In an embodiment, the surgical instrument is a single use sterilized surgical instrument. In an embodiment, the surgical instrument is configured to be usable after an exposure to an ultraviolet light surgical sterilization. In an embodiment, the surgical instrument is configured to be usable after an exposure to an autoclave or chemiclave surgical sterilization.

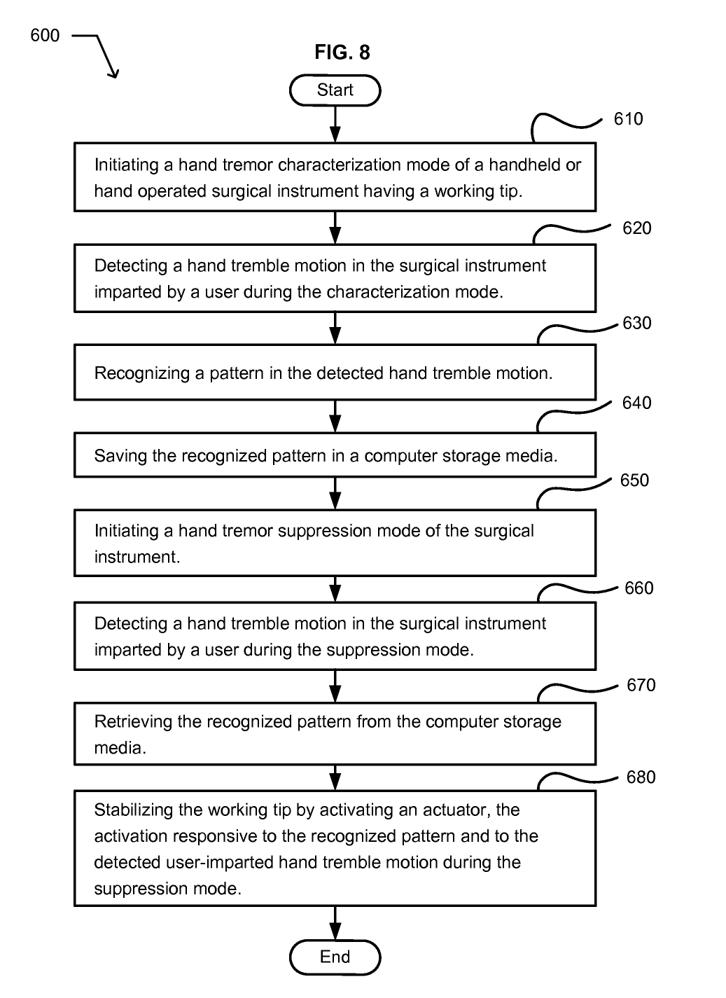

FIG. 8 illustrates an operational flow 600. After a start operation, the operational flow includes a first initiating operation 610. The first initiating operation includes initiating a hand tremor characterization mode of a handheld or hand operated surgical instrument having a working tip. In an embodiment, the first initiating operation may be implemented using the mode controller 570 of the surgical instrument 505 described in conjunction with FIG. 7. A first sensing operation 620 includes detecting a hand tremble motion in the surgical instrument imparted by a user during the characterization mode. In an embodiment, the first sensing operation may be implemented using the sensor 540 described in conjunction with FIG. 7. A recognition operation 630 includes recognizing a pattern in the detected hand tremble motion. In an embodiment, the recognition operation may be implemented using the pattern recognition module 542 described in conjunction with FIG. 7. A storage operation 640 includes saving the recognized pattern in computer storage media. In an embodiment, the storage operation may be implemented using the computer storage media 580 described in conjunction with FIG. 7. A second initiating operation 650 includes initiating a hand tremor suppression mode of the surgical instrument. In an embodiment, the second state initiating operation may be implemented using the mode controller 570 described in conjunction with FIG. 7. A second detecting operation 660 includes detecting a hand tremble motion in the surgical instrument imparted by a user during the suppression mode. In an embodiment, the second detecting operation may be implemented using the sensor 540 described in conjunction with FIG. 7. A fetching operation 670 includes retrieving the recognized pattern from the computer storage media. In an embodiment, the fetching operation may be implemented by the controller 560 fetching the recognized pattern from the computer storage media 580 described in conjunction with FIG. 7. A steadying operation 680 includes stabilizing the working tip by activating an actuator. The activation is responsive to the recognized pattern and to the detected user-imparted hand tremble motion during the suppression mode. In an embodiment, the stabilizing includes stabilizing the working tip in at least one degree of freedom. In an embodiment, the steading operation may be implemented by the controller 560 described in conjunction with FIG. 7. The operational flow includes an end operation.

In an embodiment of the first state activation 610, the initiating a hand tremor characterization mode includes initiating a hand tremor characterization mode of a surgical instrument in response to a received selection. In an embodiment of the first state activation, the initiating includes initiating a hand tremor characterization mode of a surgical instrument in response to an input received from the user. In an embodiment of the recognition operation 630, the recognizing includes recognizing a pattern in the detected user-imparted hand tremble motion occurring over a period of time. In an embodiment of the storage operation 640, the saving includes saving the recognized pattern in a local or a remote computer storage media. For example, FIG. 1 illustrates a third-party device 298 that includes a computer storage media. In an embodiment of the second state initiating 650, the initiating a hand tremor suppression mode includes initiating a hand tremor suppression mode of the surgical instrument in response to a received selection. In an embodiment of the second state activation, the initiating a hand tremor suppression mode includes initiating a hand tremor suppression mode of the surgical instrument in response to an input received from the user.

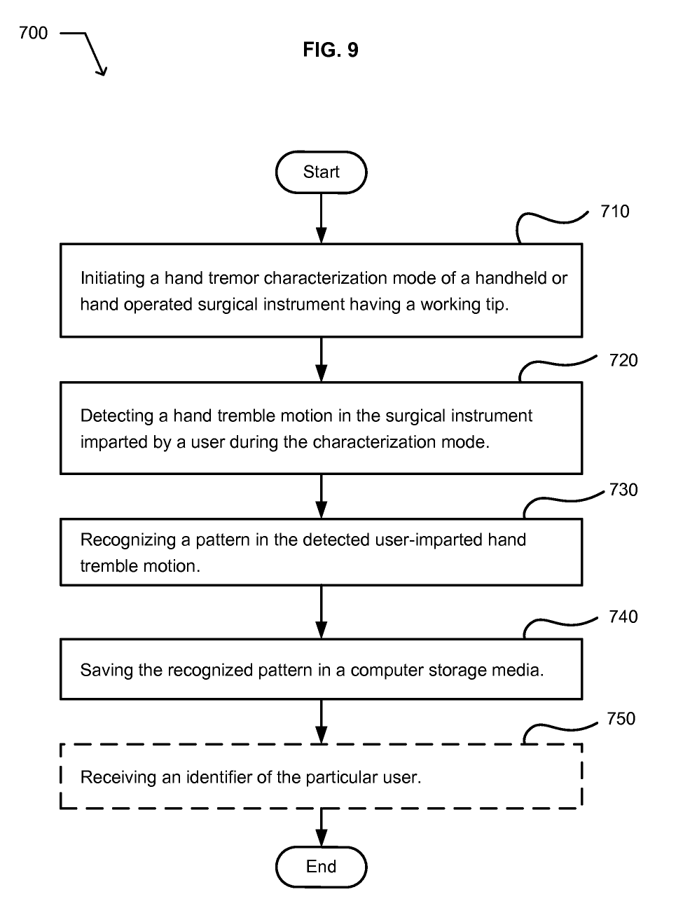

FIG. 9 illustrates an example operational flow 700 characterizing a hand tremor motion created by a particular user in a handheld or hand operated surgical instrument. After a start operation, the operational flow includes an initiating operation 710. The initiating operation includes initiating a hand tremor characterization mode of a handheld or hand operated surgical instrument having a working tip. In an embodiment, the initiating operation may be implemented using the mode controller 570 of the surgical instrument 505 described in conjunction with FIG. 7. A sensing operation 720 includes detecting a hand tremble motion in the surgical instrument imparted by a user during the characterization mode. In an embodiment, the sensing operation may be implemented using the sensor 540 described in conjunction with FIG. 7. A recognition operation 730 includes recognizing a pattern in the detected user-imparted hand tremble motion. In an embodiment, the recognition operation may be implemented using the pattern recognition module 542 described in conjunction with FIG. 7. A storage operation 740 includes saving the recognized pattern in a computer storage media. In an embodiment, the storage operation may be implemented using the computer storage media 580 described in conjunction with FIG. 7. The operational flow includes an end operation.

In an embodiment, the operational flow 700 may include at least one additional operation. An additional operation may include issuing a warning if the recognized pattern exceeds a threshold level of tremor severity. For example, a warning may be issued to the user. For example, the warning to the user may be transmitted using an audio, haptic, light device. For example, a warning may be issued to third party, such as a nurse, or a supervisor. An additional operation may include inactivating an aspect of the surgical instrument if the recognized pattern exceeds a threshold level of tremor severity. For example, inactivating may include deactivate power to the surgical instrument, or rendering the working tip not usable.

An additional operation includes receiving an identifier of the particular user. In an embodiment of the storage operation 740, the saving includes saving in computer storage media the recognized pattern in an association with an identifier of the particular user. In an embodiment, the computer storage media is a component of the surgical instrument. In an embodiment, the computer storage media is remote from the surgical instrument.

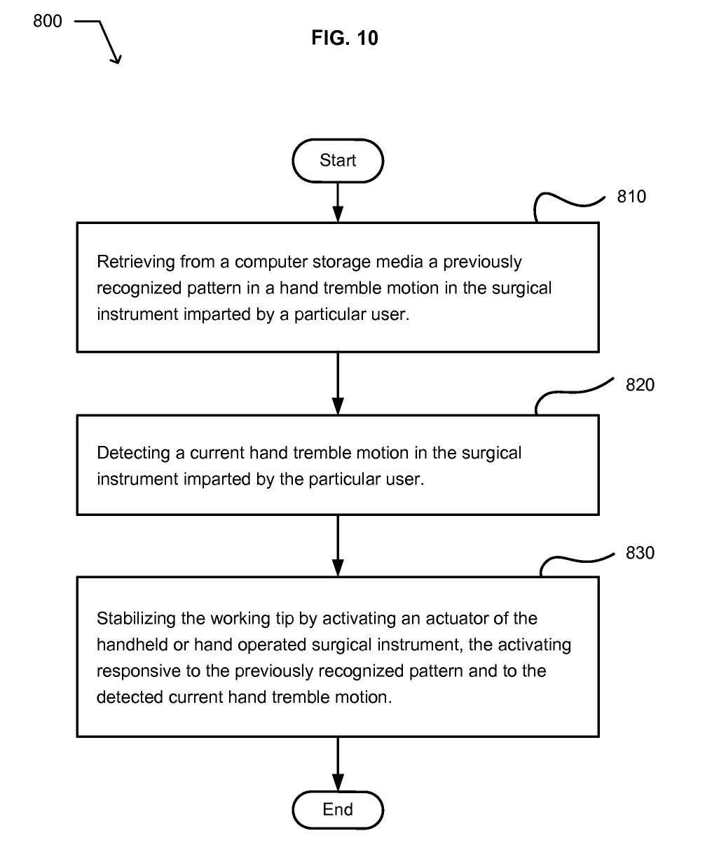

FIG. 10 illustrates an example operational flow 800 of stabilizing a working tip of a handheld or hand operated surgical instrument with respect to a hand tremor motion created by a particular user. After a start operation, the operational flow includes a fetching operation 810. The fetching operation includes retrieving from a computer storage media a previously recognized pattern in a hand tremble motion in the surgical instrument imparted by a particular user. In an embodiment, the fetching operation may be implemented by the controller 560 fetching the recognized pattern from the computer storage media 580 described in conjunction with FIG. 7. A sensing operation 820 includes detecting a current hand tremble motion in the surgical instrument imparted by the particular user. In an embodiment, the sensing operation may be implemented using the sensor 540 described in conjunction with FIG. 7. A steadying operation 830 includes stabilizing the working tip by activating an actuator of the handheld or hand operated surgical instrument. The activating is responsive to the previously recognized pattern and to the detected current hand tremble motion. In an embodiment, the steading operation may be implemented by the controller 560 described in conjunction with FIG. 7. The operational flow includes an end operation.

In an embodiment of the fetching operation 810, the retrieving includes retrieving a recognized pattern in a hand tremble motion imparted by the particular user in the surgical instrument and saved in an association with an identifier of the particular user. In an embodiment, the operational flow 800 may include at least one additional operation. An additional operation includes initiating a hand tremor suppression mode of the surgical instrument.

All references cited herein are hereby incorporated by reference in their entirety or to the extent their subject matter is not otherwise inconsistent herewith.

In some embodiments, “configured” includes at least one of designed, set up, shaped, implemented, constructed, or adapted for at least one of a particular purpose, application, or function.

It will be understood that, in general, terms used herein, and especially in the appended claims, are generally intended as “open” terms. For example, the term “including” should be interpreted as “including but not limited to.” For example, the term “having” should be interpreted as “having at least.” For example, the term “has” should be interpreted as “having at least.” For example, the term “includes” should be interpreted as “includes but is not limited to,” etc. It will be further understood that if a specific number of an introduced claim recitation is intended, such an intent will be explicitly recited in the claim, and in the absence of such recitation no such intent is present. For example, as an aid to understanding, the following appended claims may contain usage of introductory phrases such as “at least one” or “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a receiver” should typically be interpreted to mean “at least one receiver”); the same holds true for the use of definite articles used to introduce claim recitations. In addition, even if a specific number of an introduced claim recitation is explicitly recited, it will be recognized that such recitation should typically be interpreted to mean at least the recited number (e.g., the bare recitation of “at least two chambers,” or “a plurality of chambers,” without other modifiers, typically means at least two chambers).

In those instances where a phrase such as “at least one of A, B, and C,” “at least one of A, B, or C,” or “an [item] selected from the group consisting of A, B, and C,” is used, in general such a construction is intended to be disjunctive (e.g., any of these phrases would include but not be limited to systems that have A alone, B alone, C alone, A and B together, A and C together, B and C together, or A, B, and C together, and may further include more than one of A, B, or C, such as A₁, A₂, and C together, A, B₁, B₂, C₁, and C₂ together, or B₁ and B₂ together). It will be further understood that virtually any disjunctive word or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The herein described aspects depict different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality. Any two components capable of being so associated can also be viewed as being “operably couplable” to each other to achieve the desired functionality. Specific examples of operably couplable include but are not limited to physically mateable or physically interacting components or wirelessly interactable or wirelessly interacting components.

With respect to the appended claims the recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Use of “Start,” “End,” “Stop,” or the like blocks in the block diagrams is not intended to indicate a limitation on the beginning or end of any operations or functions in the diagram. Such flowcharts or diagrams may be incorporated into other flowcharts or diagrams where additional functions are performed before or after the functions shown in the diagrams of this application. Furthermore, terms like “responsive to,” “related to,” or other past-tense adjectives are generally not intended to exclude such variants, unless context dictates otherwise.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A handheld or hand operated surgical instrument comprising:
an elongated member having a longitudinal axis and a handle portion configured to be gripped or held by a user;
a working tip coupled to the elongated member;
a sensor configured to detect a hand tremble motion in the elongated member of the surgical instrument imparted by the user;
a mode controller configured to receive a selection of a hand tremor characterization mode of the surgical instrument or a hand tremor suppression mode of the surgical instrument;
a pattern recognition module configured to recognize a pattern in the detected hand tremble motion in the elongated member imparted by the user if the hand tremor characterization mode is selected;
a flexible beam element of the elongated member configured to reversibly bend, extend, or rotate with respect to the longitudinal axis;
an actuator physically coupled to the flexible beam element and configured to reversibly bend, extend, or rotate the flexible beam element with respect to the longitudinal axis;
a controller configured to stabilize the working tip by activating the actuator if the hand tremor suppression mode is selected, the activating responsive to the recognized pattern and to the detected user-imparted hand tremble motion during the suppression mode.

2. The surgical instrument of claim 1, wherein the flexible beam element of the elongated member is located in-between the handle portion and the working tip.

3. The surgical instrument of claim 1, wherein the mode controller is further configured to receive a user selection of the hand tremor characterization mode of the surgical instrument and an identifier of the user.

4. The surgical instrument of claim 1, wherein the pattern recognition module is configured to associate the identifier of the user with the recognized pattern in the user-imparted hand tremble motion.

5. The surgical instrument of claim 1, wherein the pattern recognition module is configured to recognize a pattern in the detected hand tremble motion in the elongated member imparted by the user occurring over a period of time.

6. The surgical instrument of claim 1, wherein the controller is further configured to control the activation of the actuator in response to a combination of the recognized pattern in the user-imparted hand tremble motion and the detected user-imparted hand tremble motion.

7. The surgical instrument of claim 6, wherein the controller is further configured to control the activation of the actuator in response to a weighted combination of the recognized pattern in the user-imparted hand tremble motion and the detected user-imparted hand tremble motion.

8. The surgical instrument of claim 1, further comprising:
a computer storage media configured to save the recognized pattern in the user-imparted hand tremble motion.

9. The surgical instrument of claim 8, wherein the computer storage media configured to save the recognized pattern in the user-imparted hand tremble motion in an association with an identifier of the user.

10. The surgical instrument of claim 1, wherein the controller is further configured to retrieve from a computer storage media the recognized pattern in the user-imparted hand tremble motion saved in an association with an identifier of the user.

11. The surgical instrument of claim 1, wherein the surgical instrument is sterilized.

12. The surgical instrument of claim 1, wherein the surgical instrument is configured to be usable after a sterilization.

13. The surgical instrument of claim 1, wherein the surgical instrument is configured to be usable after an exposure to one sterilization for a surgical use.

14. The surgical instrument of claim 13, wherein the surgical instrument is configured to be usable after an exposure to a surgical sterilization condition.

15. The surgical instrument of claim 1, wherein the surgical instrument is a single use sterilized surgical instrument.

16. The surgical instrument of claim 1, wherein the surgical instrument is configured to be usable after an exposure to an ultraviolet light surgical sterilization.

17. The surgical instrument of claim 1, wherein the surgical instrument is configured to be usable after an exposure to an autoclave or chemiclave surgical sterilization.

18. A method comprising:
initiating a hand tremor characterization mode of a handheld or hand operated surgical instrument having a working tip;
detecting a hand tremble motion in the surgical instrument imparted by a user during the characterization mode;
recognizing a pattern in the detected hand tremble motion;
saving the recognized pattern in a computer storage media;
initiating a hand tremor suppression mode of the surgical instrument;
detecting a hand tremble motion in the surgical instrument imparted by a user during the suppression mode;
retrieving the recognized pattern from the computer storage media; and
stabilizing the working tip by activating an actuator, the activation responsive to the recognized pattern and to the detected user-imparted hand tremble motion during the suppression mode.

19. The method of claim 18, wherein the initiating a hand tremor characterization mode includes initiating a hand tremor characterization mode of a surgical instrument in response to a received selection.

20. The method of claim 18, wherein the initiating includes initiating a hand tremor characterization mode of a surgical instrument in response to a received input from the user.

21. The method of claim 18, wherein the recognizing includes recognizing a pattern in the detected user-imparted hand tremble motion occurring over a period of time.

22. The method of claim 18, wherein the saving includes saving the recognized pattern in a local or a remote computer storage media.

23. The method of claim 18, wherein the initiating a hand tremor suppression mode includes initiating a hand tremor suppression mode of the surgical instrument in response to a received selection.

24. The method of claim 18, wherein the initiating a hand tremor suppression mode includes initiating a hand tremor suppression mode of the surgical instrument in response to an input received from the user.

25. A method of characterizing a hand tremor motion created by a particular user in a handheld or hand operated surgical instrument, the method comprising:
initiating a hand tremor characterization mode of a handheld or hand operated surgical instrument having a working tip;
detecting a hand tremble motion in the surgical instrument imparted by a user during the characterization mode;
recognizing a pattern in the detected user-imparted hand tremble motion; and
saving the recognized pattern in a computer storage media.

26. The method of claim 25, further comprising:
issuing a warning if the recognized pattern exceeds a threshold level of tremor severity.

27. The method of claim 25, further comprising:
inactivating an aspect of the surgical instrument if the recognized pattern exceeds a threshold level of tremor severity.

28. The method of claim 25, further comprising:
receiving an identifier of the particular user.

29. The method of claim 25, wherein the saving includes saving in the computer storage media the recognized pattern in an association with an identifier of the particular user.

30. The method of claim 25, wherein the computer storage media is a component of the surgical instrument.

31. The method of claim 25, wherein the computer storage media is remote from the surgical instrument.

32. A method of stabilizing a working tip of a handheld or hand operated surgical instrument with respect to a hand tremor motion created by a particular user in, the method comprising:
retrieving from a computer storage media a previously recognized pattern in a hand tremble motion in the surgical instrument imparted by a particular user;
detecting a current hand tremble motion in the surgical instrument imparted by the particular user; and
stabilizing the working tip by activating an actuator of the handheld or hand operated surgical instrument, the activating responsive to the previously recognized pattern and to the detected current hand tremble motion.

33. The method of claim 32, wherein the retrieving includes retrieving a recognized pattern in a hand tremble motion imparted by the particular user in the surgical instrument and saved in an association with an identifier of the particular user.

34. The method of claim 32, further comprising:
initiating a hand tremor suppression mode of the surgical instrument.
